(19) United States
(12) Patent Application Publication
(10) Pub. No.: US 2022/0351845 A1
(43) Pub. Date: Nov. 3, 2022

(54) DEMENTIA PATIENT INTEGRATED MANAGEMENT SYSTEM

(71) Applicant: C2 MONSTER CO., LTD., Seoul (KR)

(72) Inventors: Jinsung Choi, Uiwang-si (KR); Sangin Kim, Jeju-si (KR); Jongho Park, Seoul (KR); Hyeongmin Kim, Jeju-si (KR)

(73) Assignee: C2 MONSTER CO., LTD., Seoul (KR)

(21) Appl. No.: 17/728,897

(22) Filed: Apr. 25, 2022

(30) Foreign Application Priority Data
Apr. 30, 2021 (KR) 10-2021-0056878
Dec. 29, 2021 (KR) 10-2021-0190657

(51) Int. Cl. G16H 40/20; G16H 40/67

(52) U.S. Cl. CPC G16H 40/20; G16H 40/67

(57) ABSTRACT

Disclosed is a dementia patient integrated management system including an information registration unit for registering manager information of a manager belonging to at least one institution and user information of a user managed by the manager, an information receiving unit for receiving real-time location information of the user, a user analysis unit for analyzing an action of the user through the location information, and an information management unit for updating the real-time location information of the user and analysis information analyzed by the user analysis unit in the user information registered in the information registration unit and managing the user information. The user information managed by the information management unit is shared such that another manager other than the manager is capable of identifying the user information. Integrated management is achieved by a plurality of managers.

### STATEMENT REGARDING GOVERNMENT SPONSORED RESEARCH OR DEVELOPMENT

The inventive concept is derived from research conducted as part of personal basic research (Ministry of Science and ICT) (R&D) (Project Identification Number: 2020000041, Research Management Institution: Institute for Information & communication Technology Planning & evaluation(IITP), Research Project Title: Development of cognitive training program (app) for overcoming dementia in the elderly and development of integrated emergency safety service based on biometric information from smart watch, Host Institution: C2Monster Corp., Research Period: 2020.04.01-2021.12.31, Contribution Rate: 1/1) of the Ministry of Science and ICT.

There is no property interest of the Korean government in any aspect of this invention.

### CROSS-REFERENCE TO RELATED APPLICATIONS

This application claims priority under 35 U.S.C. § 119 to Korean Patent Application No. 10-2021-0056878 filed on Apr. 30, 2021, and Korean Patent Application No. 10-2021-0190657 filed on Dec. 29, 2021, in the Korean Intellectual Property Office, the disclosures of which are incorporated by reference herein in their entireties.

### BACKGROUND

Embodiments of the present disclosure described herein relate to a dementia patient integrated management system.

With the shift to an aging society, a need for management of the elderly is increasing significantly. In particular, in case of an emergency for the elderly living alone, when no action is taken, the elderly are at great risk, which is a major social question.

In particular, the management of the elderly is more important due to senile dementia. To manage dementia patients, local governments have introduced and operated a system for caring for dementia patients, for example, periodically dispatching a life manager or social worker to the home of a dementia patient.

However, a system using manpower is greatly inefficient due to lack of budget and time.

Moreover, when a dementia patient goes missing, because the activity pathway of the dementia patient has not been grasped, the search time of the dementia patient may increase. When the search is delayed, unexpected accidents may occur due to environmental factors.

### SUMMARY

Embodiments of the present disclosure provide a dementia patient integrated management system capable of collectively determining and training cognitive abilities through pieces of user information by registering and managing manager information of a manager belonging to at least one or more institutions and user information of a user managed by the manager, and by sharing the user information such that another manager other than the manager is capable of identifying the user information.

Moreover, embodiments of the present disclosure provide a dementia patient integrated management system capable of promptly responding to abnormal symptoms such as disappearance of dementia patients and departure from a safe zone, by variously analyzing a user's action by using location information and identifying environment information.

According to an embodiment, a dementia patient integrated management system includes an information registration unit for registering manager information of a manager belonging to at least one institution and user information of a user managed by the manager, an information receiving unit for receiving real-time location information of the user, a user analysis unit for analyzing an action of the user through the location information, and an information management unit for updating the real-time location information of the user and analysis information analyzed by the user analysis unit in the user information registered in the information registration unit and managing the user information. The user information managed by the information management unit is shared such that another manager other than the manager is capable of identifying the user information. Integrated management is achieved by a plurality of managers.

Furthermore, the user analysis unit analyzes an action during a predetermined time through movement analysis of analyzing whether the user moves, based on the location information and moving line analysis of tracking and analyzing a moving line, along which the user wanders, through the movement analysis.

Moreover, the information management unit sets a safe zone, which is based on the moving line analyzed by the user analysis unit or a set address of the user. The user analysis unit further performs speed analysis of analyzing a walking speed of the user through the movement analysis and the moving line analysis and transportation means analysis of analyzing whether the user is moving on foot or through a transportation means, through the speed analysis. When the user deviates from a predetermined safe distance from the safe zone, the dementia patient integrated management system further includes a call unit that provides a caution warning to the manager when it is determined that the user is walking, through the transportation means analysis or provides a hazard warning to the manager when the user is moving through the transportation means, the user reaches a location within a predetermined setting distance, at which a return is impossible, after the caution warning, or the user fails to move to the safe zone within a predetermined first set time after the caution warning.

Also, the user analysis unit extracts the moving line, on which the user is walking and which is identified through the transportation means analysis, analyzes an daily exercise amount through the extracted moving line, analyzes an average exercise amount based on at least two daily exercise amounts thus already analyzed, and analyzes whether the daily exercise amount is satisfied, by comparing the daily exercise amount with the average exercise amount. The call unit provides the caution warning to the manager when the daily exercise amount is not satisfied, and provides the hazard warning to the manager when the daily exercise amount is less than a minimum exercise amount among the daily exercise amounts thus already analyzed.

Besides, the user analysis unit analyzes a patient wandering pattern through location analysis and moving line analysis of a patient having mild cognitive impairment, and analyzes a user wandering pattern through location analysis and moving line analysis of the user. When a similarity between the patient wandering pattern and the user wandering pattern is not less than a predetermined value, the information management unit updates mild cognitive impairment information in the user information of the user. The call unit selects a call case through the user information about the user, and provides the manager with the hazard warning instead of the caution warning when it is determined that the user has mild cognitive impairment.

In addition, the user analysis unit analyzes a stop time at a place, at which the user is stopped, through the location information. The dementia patient integrated management system further includes an environment information receiving unit for requesting and receiving environment information included in location information of the place, at which the user is stopped, from Korea Meteorological Administration when the stop time reaches a predetermined second set time. The call unit provides the manager with the caution warning when the stop time is not less than a predetermined third set time, and provides the manager with the caution warning when a temperature in the environment information received by the environment information receiving unit is out of a predetermined temperature range or an air quality value in the environment information is out of a predetermined air quality value.

Furthermore, the dementia patient integrated management system further includes a check unit for attempting to make a phone call between a terminal of the manager and a mobile terminal of the user when the caution warning or the hazard warning is received from the call unit, a path extraction unit for determining disappearance, when the phone call attempted through the check unit is not connected, to extract a missing search path, and extracting a past user wandering pattern identified within a predetermined search distance, based on latest location information of a missing person who is determined to be missing, and a search information transmitting unit for delivering place information of a stop place in the user wandering pattern extracted by the path extraction unit to a searcher such that the searcher intensively searches for the missing person at a location corresponding to the place information.

Moreover, the search information transmitting unit transmits a photo and characteristic information of the missing person to at least one searcher terminal. The searcher terminal is a terminal of a delivery person, which has an agreement with the institution and which is positioned in a specific radius based on the stop place. When a photo or video of the missing person is captured, the searcher terminal transmits the photo or video to the terminal of the manager and automatically deletes the photo and characteristic information of the missing person when it is identified that the missing person is correct, by the terminal of the manager. The dementia patient integrated management system further includes a reward delivery unit for delivering an agreed reward to the searcher terminal, which finds the missing person, when the missing person is taken over to the manager.

### BRIEF DESCRIPTION OF THE FIGURES

The above and other objects and features of the present disclosure will become apparent by describing in detail embodiments thereof with reference to the accompanying drawings.

FIG. 1 is a diagram illustrating an example of a terminal, to which a dementia patient integrated management system is capable of being applied, according to an embodiment of the present disclosure.

FIG. 2 is a diagram illustrating a detailed configuration of a sensing unit of FIG. 1.

FIGS. 3 and 4 are block diagrams illustrating a dementia patient integrated management system, according to an embodiment of the present disclosure.

FIGS. 5 and 6 are views for analyzing actions according to a user analysis unit of a dementia patient integrated management system, according to an embodiment of the present disclosure.

FIG. 7 is a diagram illustrating a cognitive training question including two or more cognitive training paths provided by a question providing unit of a dementia patient integrated management and dementia determination system, according to an embodiment of the present disclosure.

FIG. 8 is a diagram from extracting a screen implemented by using an application program of a dementia patient integrated management and dementia determination system, according to an embodiment of the present disclosure.

### DETAILED DESCRIPTION

The above and other aspects, features and advantages of the present disclosure will become apparent from the following description of the following embodiments given in conjunction with the accompanying drawings. However, the present disclosure is not limited to the embodiments disclosed below, but may be implemented in various forms. The embodiments of the present disclosure is provided to make the disclosure of the present disclosure complete and fully inform those skilled in the art to which the present disclosure pertains of the scope of the present disclosure. The same reference numerals denote the same elements throughout the specification.

Hereinafter, the present disclosure will be described with reference to drawings for describing a dementia patient integrated management system according to embodiments of the present disclosure.

First of all, a user may possess a mobile terminal 100 to communicate with the dementia patient integrated management system according to an embodiment of the present disclosure and to improve a user's cognitive training and cognitive ability.

Moreover, a manager may identify location information through the user's mobile terminal 100, may manage the user, and may collectively manage the user through sharing the location information with another manager.

As such, to implement a dementia patient integrated management system according to an embodiment of the present disclosure, the mobile terminal 100 carried by the user will be described in detail.

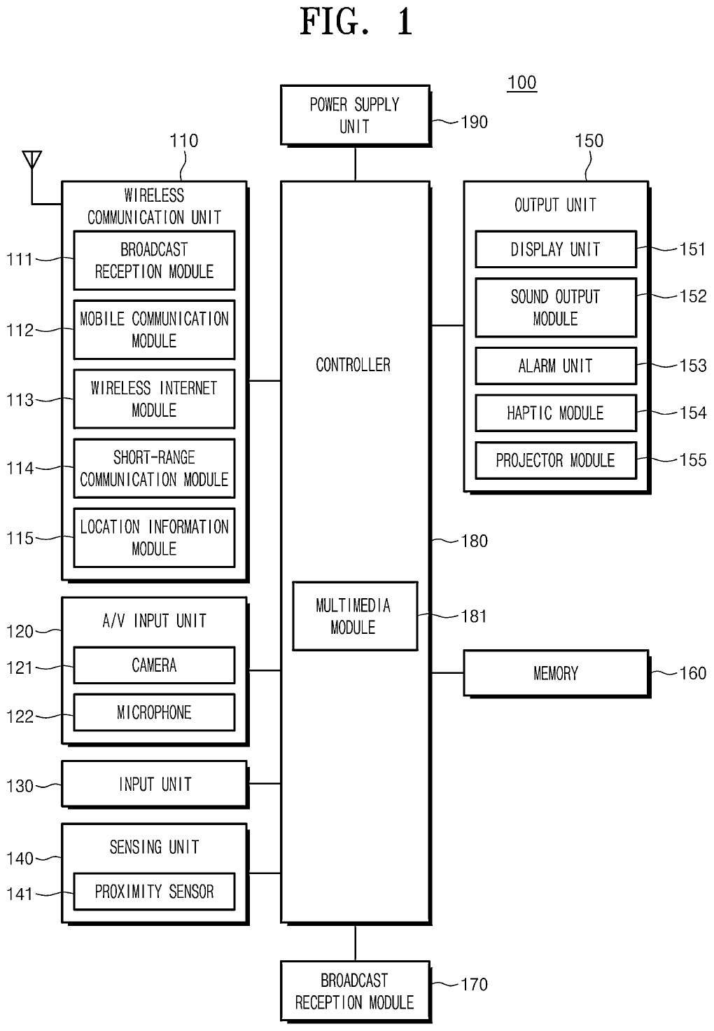

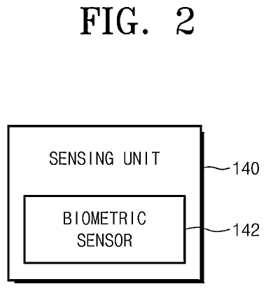

FIG. 1 is a diagram illustrating an example of a terminal, to which a dementia patient integrated management system is capable of being applied, according to an embodiment of the present disclosure. FIG. 2 is a diagram illustrating a detailed configuration of a sensing unit of FIG. 1.

Referring to FIG. 1, the mobile terminal 100 in which dementia patient integrated management system is implemented may include a wireless communication unit 110, an audio/video (A/V) input unit 120, an input unit 130, a sensing unit 140, an output unit 150, a memory 160, an interface unit 170, a controller 180, and a power supply unit 190.

However, the components illustrated in FIG. 1 are not essential. For example, a data transmission device having more or fewer components than the components illustrated in FIG. 1 may be implemented.

Hereinafter, the components will be described in order.

The wireless communication unit 110 may include one or more modules that enable wireless communication between the mobile terminal 100 and a wireless communication system or between the mobile terminal 100 and a network in which local communication is located. For example, the wireless communication unit 110 may include a broadcast reception module 111, a mobile communication module 112, a wireless Internet module 113, a short-range communication module 114, and a location information module 115.

For example, the wireless communication unit 110 may transmit information about whether a user's cognitive ability is improved or decreased, at a request of the outside or at a predetermined period.

Here, the outside may be a doctor, manager, or guardian who remotely determines the user's status and requests information for treatment.

That is, a doctor or manager may receive information about whether the user's cognitive ability is improved or decreased, at a predetermined period or in real time by using the dementia patient integrated management system provided in the present disclosure, may determine the user's status according to the information, and may take measures.

The broadcast reception module 111 receives a broadcast signal and/or broadcast-related information from an external broadcast management server through a broadcast channel.

The broadcast channel may include a satellite channel and a terrestrial channel. A broadcast management server may refer to a server that generates and transmits a broadcast signal and/or broadcast-related information or a server that receives a broadcast signal and/or broadcast-related information, which is previously generated, and transmits the received signal or information to a data transmission device. In addition to a TV broadcast signal, a radio broadcast signal, and a data broadcast signal, the broadcast signal may include a broadcast signal in which a TV broadcast signal or a radio broadcast signal is combined with a data broadcast signal.

The broadcast-related information may mean information related to a broadcast channel, a broadcast program, or a broadcast service provider.

The broadcast-related information may be provided through a mobile communication network. In this case, the broadcast-related information may be received by the mobile communication module 112.

The broadcast-related information may be present in various forms. For example, the broadcast-related information may be present in a form of an electronic program guide (EPG) of digital multimedia broadcasting (DMB) or an electronic service guide (ESG) of digital video broadcast-handheld (DVB-H).

For example, the broadcast reception module 111 may receive a digital broadcast signal by using a digital broadcasting system such as digital multimedia broadcasting-terrestrial (DMB-T), digital multimedia broadcasting-satellite (DMBS), media forward link only (MediaFLO), digital video broadcast-handheld (DVB-H), DVB-CBMS, OMA-BCAST, integrated services digital broadcast-terrestrial (ISDB-T), or the like. In addition, the broadcast reception module 111 may be implemented to be suitable for another broadcasting system as well as the above-described digital broadcasting system.

The broadcast signal and/or broadcast-related information received through the broadcast reception module 111 may be stored in the memory 160.

The mobile communication module 112 transmits and receives a wireless signal with at least one of a base station on a mobile communication network, an external data transmission device, and a server. The wireless Internet module 113 refers to a module for wireless Internet access, and may be built in the mobile terminal 100 or mounted on the mobile terminal 100.

Wireless LAN (WLAN) (Wi-Fi), wireless broadband (Wibro), world interoperability for microwave access (Wimax), or high speed downlink packet access (HSDPA) may be used as a wireless Internet technology.

The short-range communication module 114 refers to a module for short range communication. Bluetooth, radio frequency identification (RFID), infrared data association (IrDA), ultra-wideband (UWB), or ZigBee may be used as a technology of short-range communication.

Besides, the short-range communication module 114 may perform the above-described visible light wireless communication.

The location information module 115 is a module for obtaining a location of the mobile terminal 100, and is a global position system (GPS) module as a representative example. According to the current technology, the GPS module may accurately calculate three-dimensional current location information according to latitude, longitude, and altitude, by calculating accurate time information and information about distances from 3 or more satellites, and applying trigonometry to the calculated information. Nowadays, a method is widely used to calculate information about a location and time by using three satellites and to correct an error of the calculated information about the location and time by using another satellite. In addition, the GPS module may calculate speed information by continuously calculating a current location in real time.

Accordingly, the location information module 115 transmits location information to an information receiving unit to be described later.

Referring to FIG. 1, the A/V input unit 120 may be used to enter an audio signal or a video signal, and may include a camera 121 and a microphone 122. The camera 121 processes an image frame, which is obtained by an image sensor in a video call mode or a shooting mode, such as a still image or a moving image. The processed image frame may be displayed on a display unit 151.

The image frame processed by the camera 121 may be stored in the memory 160 or may be transmitted to the outside through the wireless communication unit 110.

At this time, the two or more cameras 121 may be provided depending on use environments.

For example, the camera 121 may include first and second cameras for taking 3D images on an opposite surface of the mobile terminal 100 on which the display unit 151 is provided, and may further include a third camera for the user's self-photography in a portion of a surface where the display unit 151 of the mobile terminal 100 is provided.

At this time, the first camera may be used to capture an image for a left eye, which is a source image of a 3D image. The second camera may be used to capture an image for a right eye, which is another source image of the 3D image.

The microphone 122 receives an external sound signal from a microphone in a call mode or recording mode, a voice recognition mode, or the like and processes the external sound signal as electrical voice data. In the call mode, the processed voice data may be converted into a format capable of being transmitted to a mobile communication base station through the mobile communication module and then may be output.

Various noise cancellation algorithms for canceling noise generated in a process of receiving an external sound signal may be implemented in the microphone 122.

The input unit 130 generates input data that allows a user to control an operation of a data transmission device.

The input unit 130 may receive, from the user, a signal for designating two or more of the displayed contents according to an embodiment of the present disclosure. In addition, a signal designating two or more contents may be received through a touch input or may be received through a hard key and a soft key input.

The input unit 130 may receive an input for selecting one or more contents from a user.

In addition, the input unit 130 may receive, from a user, an input for generating an icon related to a function capable of being performed by the mobile terminal 100.

As described above, the input unit 130 may be composed of a direction key, a key pad, a dome switch, a touch pad (static pressure/capacitance), a jog wheel, a jog switch, and the like.

Besides, the sensing unit 140 generates a sensing signal for controlling an operation of the mobile terminal 100 by sensing a current state of the mobile terminal 100, such as an open/close status of the mobile terminal 100, a location of the mobile terminal 100, whether a user is contacted, an orientation of a data transmission device, acceleration/deceleration of the data transmission device, or the like. Furthermore, the sensing unit 140 may sense whether the power of the power supply unit 190 is supplied, or whether the interface unit 170 is coupled to an external device. In the meantime, the sensing unit 140 may include a proximity sensor 141.

Meanwhile, referring to FIG. 2, the sensing unit 140 may sense the user's biometric information including at least one of pupil information, pressure information, voice information, pulse information, and body temperature information.

That is, the sensing unit 140 may sense the user's pupil information, pressure information, voice information, pulse information, body temperature information, pulse, and the like, and may convert the sensed information into a digital signal.

For example, the sensing unit 140 may generate the user's pupil size information by continuously sensing the user's pupil pulse information, and may store the user's pupil size information in the memory 160.

Here, the sensing unit 140 may include the sensed time information in the sensed biometric information of the user and may store the biometric information of the user in the memory 160.

In the meantime, the sensing unit 140 may analyze a portion of the user information entered through the A/V input unit 120 and the input unit 130 as a biological signal and may convert the biological signal into a digital signal.

The output unit 150 may periodically output a plurality of questions or pieces of information provided through the controller 180 and may provide the user with the plurality of questions or the pieces of information.

Here, the output unit 150 may provide a question or information in a visual, auditory or tactile manner.

The output unit 150 may generate an output related to visual, auditory, or tactile sensation, and may include the display unit 151, a sound output module 152, an alarm unit 153, a haptic module 154, and a projector module 155.

The display unit 151 displays (outputs) information processed by the mobile terminal 100. For example, when the data transmission device is in a call mode, the display unit 151 displays a user interface (UI) or graphic user interface (GUI) related to a call. When the mobile terminal 100 is in a video call mode or shooting mode, the display unit 151 displays the captured and/or the received image, UI, or GUI.

In addition, the display unit 151 according to an embodiment of the present disclosure supports 2D and 3D display modes.

That is, the display unit 151 according to an embodiment of the present disclosure may have a configuration in which a switch liquid crystal is coupled to a general display device. Besides, the direction of light may be controlled such that different rays reach left and right eyes, by operating an optical parallax barrier by using the switch liquid crystal. Accordingly, when an image from combining a right-eye image and a left-eye image is displayed on a display device, the user sees an image corresponding to each eye and feels as if the image was displayed in three dimensions.

That is, in a 2D display mode, under the control of the controller 180, the display unit 151 performs a general 2D display operation by driving only the display device without driving the switch liquid crystal and the optical parallax barrier.

Furthermore, in a 3D display mode, under the control of the controller 180, the display unit 151 performs a 3D display operation by driving only the display device 151a with driving the switch liquid crystal, the optical parallax barrier 50, and the display device.

In the meantime, as described above, the display unit 151 may include at least one of a liquid crystal display (LCD), a thin film transistor-liquid crystal display (TFT-LCD), an organic light emitting diode (OLED), a flexible display, or a 3D display.

A part of these displays may be implemented with a transparent display or a light-transmitting display such that a user sees the outside through the part of these displays. This may be called a transparent display, and a typical example of the transparent display includes a transparent OLED (TOLED). The rear structure of the display unit 151 may also be implemented as a light transmitting structure. The user may see an object located behind the data transmission device through a region occupied by the display unit 151 of a body of the data transmission device by using this structure.

Two or more of the display units 151 may be present depending on the implementation form of the mobile terminal 100. For example, a plurality of display units in the mobile terminal 100 may be spaced from one another on one surface or may be arranged integrally with one another. Also, the plurality of display units may be disposed on different surfaces, respectively.

When the display unit 151 and a sensor (hereinafter referred to as a ‘touch sensor’) sensing a touch operation are implemented in a mutual layer structure (hereinafter referred to as a ‘touch screen’), the display unit 151 may be used as an input device in addition to an output device. For example, the touch sensor may have a shape such as a touch film, a touch sheet, a touch pad.

That is, the input unit 130 and the output unit 150 may be implemented as a single touch screen.

The touch sensor may be configured to convert pressure applied to a specific portion of the display unit 151 or a change in capacitance occurring in a specific portion of the display unit 151 into an electrical input signal. The touch sensor may be configured to detect not only a location and area to be touched, but also the pressure at a point in time when a touch is made.

When there is a touch input to the touch sensor, the signal(s) corresponding to the touch input is sent to a touch controller (not shown). The touch controller processes the signal(s) and then sends the corresponding data to the controller 180. Accordingly, the controller 180 may determine which region of the display unit 151 has been touched.

The proximity sensor 141 may be positioned in an inner region of the data transmission device covered by the touch screen or in the vicinity of the touch screen. The proximity sensor 141 refers to a sensor that detects whether there is an object approaching a predetermined detection surface or whether there is an object present in the vicinity, by using the power of an electromagnetic field or infrared light without mechanical contacts. The proximity sensor 141 has a lifespan longer than a contact sensor and has high utility.

For example, the proximity sensor 141 includes a transmissive photoelectric sensor, a direct reflection photoelectric sensor, a mirror reflection photoelectric sensor, a high-frequency oscillation proximity sensor, a capacitive proximity sensor, a magnetic proximity sensor, and an infrared proximity sensor. When the touch screen is capacitive, the touch screen is configured to detect the proximity of a pointer by using a change in electric field according to the proximity of the pointer. In this case, a touch screen (a touch sensor) may be classified as a proximity sensor.

Hereinafter, for convenience of description, an action for recognizing a location of a pointer on the touch screen by approaching the pointer on the touch screen without touching the touch screen is referred to as a “proximity touch”. An action of actually touching the pointer on the touch screen is referred to as a “contact touch”. The location where a proximity touch is made on a touch screen by using a pointer refers to a location where the pointer vertically corresponds to the touch screen when the pointer is touched by proximity.

The proximity sensor senses a proximity touch and a proximity touch pattern (e.g.., a proximity touch distance, a proximity touch direction, a proximity touch speed, a proximity touch time, a proximity touch location, a proximity touch movement status, or the like). Information corresponding to the detected proximity touch operation and proximity touch pattern may be output on the touch screen.

In a call signal reception mode, recording mode, voice recognition mode, broadcast reception mode, or the like, the sound output module 152 may output audio data received from the wireless communication unit 110 or stored in the memory 160. The sound output module 152 also outputs a sound signal related to a function performed by the mobile terminal 100. The sound output module 152 may include a receiver, a speaker, a buzzer, and the like.

The alarm unit 153 outputs a signal for providing a notification of the occurrence of an event of the mobile terminal 100.

The alarm unit 153 may output a signal for providing a notification of the occurrence of an event in a form of, for example, vibration other than a form of a video signal or an audio signal. The video signal or audio signal may also be output through the display unit 151 or the sound output module 152. In this case, the display unit 151 and the sound output module 152 may be classified as a kind of the alarm unit 153.

Here, the user may recognize the start and end at a point in time when a question is solved, through the alarm unit 153.

The haptic module 154 generates various tactile effects that the user is capable of feeling. A representative example of the tactile effect generated by the haptic module 154 is vibration. The intensity and pattern of vibration generated by the haptic module 154 may be controlled. For example, different vibrations may be synthesized and output or may be output sequentially.

In addition to the vibration, the haptic module 154 may generate various tactile effects such as an effect of stimulation of a pin arrangement that vertically moves on a contact skin surface, blowing force or suction force of air through a nozzle or suction port, rubbing against a skin surface, a contact of an electrode, electrostatic force, or the like, or an effect by the coolness and warmth generated by using elements capable of absorbing heat or generate heat.

The haptic module 154 may not only deliver a tactile effect through a direct contact, and may but also be implemented such that the user is capable of feeling a tactile effect through a muscle sense of a finger or arm. The two or more haptic modules 154 may be provided depending on a configuration aspect of the mobile terminal 100.

The projector module 155 may be a component for performing an image project function by using the mobile terminal 100 and may display an image, which is the same as or at least partially different from the image displayed on the display unit 151, on an external screen or wall depending on the control signal of the controller 180.

In detail, the projector module 155 may include a light source (not shown) that generates light (e.g., laser light) for outputting an image to the outside, an image generating means (not shown) for generating an image to be output to the outside by using the light generated by the light source, and a lens (not shown) for magnifying and outputting an image to the outside at a specific focal length. Furthermore, the projector module 155 may include a device (not shown) capable of adjusting an image projection direction by mechanically moving the entire lens or module.

The projector module 155 may be classified into a cathode ray tube (CRT) module, a liquid crystal display (LCD) module, and a digital light processing (DLP) module depending on the device type of a display means. In particular, the DLP module may refer to a method of magnifying and projecting an image, which is generated as light generated from a light source is reflected by a digital micromirror device (DMD) chip, and may be advantageous for miniaturization of the projector module 155.

Preferably, the projector module 155 may be provided in a longitudinal direction on the side surface, front surface, or rear surface of the mobile terminal 100. In addition, the projector module 155 may be provided at any location of the mobile terminal 100, as necessary.

The memory 160 may store a program for the processing and control of the controller 180 and may perform a function for temporarily storing pieces of input/output data. The memory 160 may also store the usage frequency of each data. Furthermore, the memory 160 may store data related to vibration and sound of various patterns output when a touch input on the touch screen is input.

Herein, the memory 160 may store a question or information, which is provided through the output unit 150, the user's answer, and whether a correct answer is present.

In the meantime, questions stored in the memory 160 may be classified into a memory group for verifying a memory cognitive ability, a concentration group for verifying a concentration cognitive ability, a calculation group for verifying a calculation cognitive ability, a space-time group for verifying a spatiotemporal cognitive ability, a thought processing group for verifying a thought processing cognitive ability, and a language improvement group for verifying a language cognitive ability.

Herein, difficulty levels of a plurality of questions stored in each of the memory group, the concentration group, the calculation ability group, the space-time group, the thought processing group, and the language group may be set in grade order.

For example, difficulty levels of the plurality of questions classified into each group may be set to level 1, level 2, level 3, level 4, and level 5.

Herein, as a level is low, the difficulty level may be relatively low. As a level is high, the difficulty level may be relatively high.

In addition, the user's answer and whether a correct answer is present may be accumulated and stored in the memory 160 for each question try thus provided daily.

As described above, the memory 160 may include a storage medium of at least one type of a flash memory type, a hard disk type, a multimedia card micro type, a card type memory (e.g., SD memory, XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, an optical disc, etc. For example, the mobile terminal 100 may operate in connection with a web storage that performs a storage function of the memory 160 on the Internet.

The interface unit 170 serves as a path for all external devices connected to the mobile terminal 100. The interface unit 170 receives data from an external device; the interface unit 170 receives power and transmits the power to each component inside the mobile terminal 100; or, data inside the mobile terminal 100 is transmitted to an external device through the interface unit 170. For example, the interface unit 170 may include a wired/wireless headset port, an external charger port, a wired/wireless data port, a memory card port, a port for connecting a device equipped with an identification module, an audio input/output (I/O) port, a video I/O port, an earphone port, and the like.

In the meantime, the interface unit 170 may serve as a path through which a separate external device for sensing the user's biometric information is connected to the mobile terminal 100.

The identification module may be a chip that stores various pieces of information for authenticating the use authentication of the mobile terminal 100 and may include a user identify module (UIM), a subscriber identify module (SIM), and a universal subscriber identity module (USIM). The device (hereinafter referred to as an ‘identification device’) equipped with an identification module may be manufactured in a form of a smart card. Accordingly, the identification device may be connected to the mobile terminal 100 through a port.

When the mobile terminal 100 is connected to an external cradle, the interface unit 170 may be a path through which power from the cradle is supplied to the mobile terminal 100 or a path through which various command signals entered from the cradle by the user are delivered to the data transmission device. The various command signals or power entered from the cradle may be operated as signals for recognizing that the data transmission device is correctly mounted on the cradle.

The controller 180 may normally control the overall operation of each configuration. The controller 180 may select a question stored in the memory 160 and may provide the question to the user through the output unit 150. After the controller 180 obtains the user's input information through the input unit 130, the controller 180 may process the user's input information or may determine the user's state.

The controller 180 may include a multimedia module 181 for playing multimedia. The multimedia module 181 may be implemented within the controller 180 or may be implemented separately from the controller 180.

As described above, the mobile terminal 100 may improve the user's cognitive training and cognitive ability. Moreover, the result of cognitive training may be received by a manager from the mobile terminal 100 or may be identified remotely by the mobile terminal 100. As a plurality of managers share information, the plurality of managers may comprehensively determine and train the user.

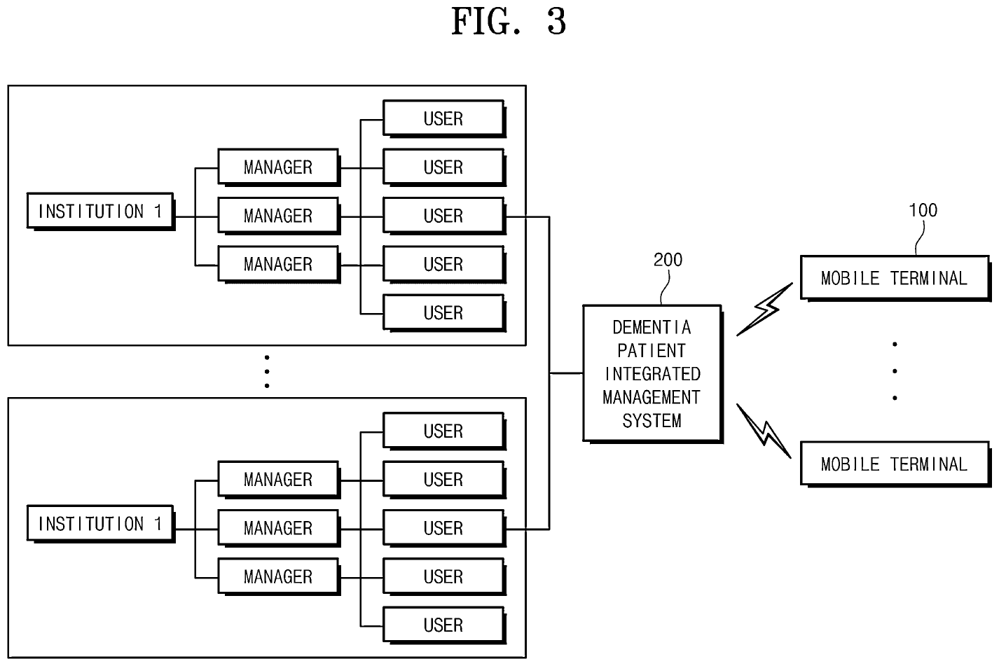

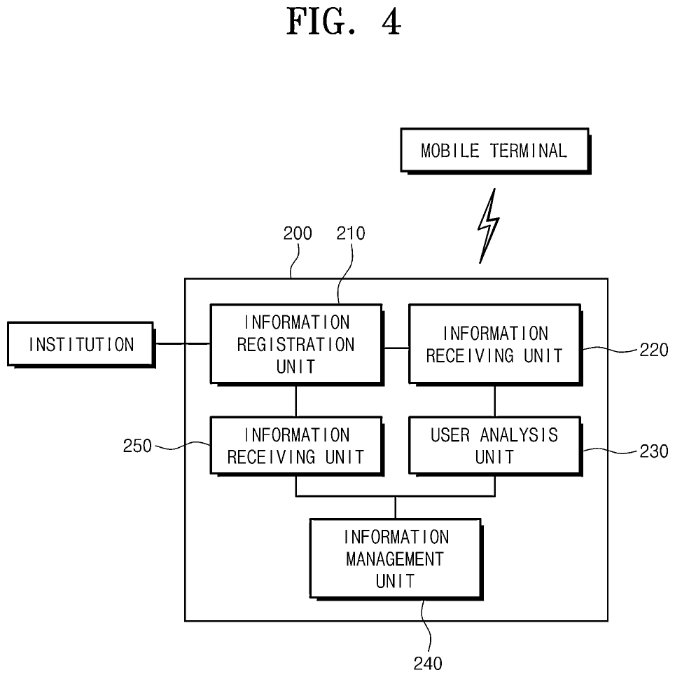

Referring to FIGS. 3 and 4, a dementia patient integrated management system 200 according to an embodiment of the present disclosure includes an information registration unit 210, an information receiving unit 220, a user analysis unit 230, an information management unit 240, and a question providing unit 250.

First of all, manager information of a manager belonging to at least one or more institutions and user information of a user managed by the manager are registered in the information registration unit 210.

At this time, there are a plurality of managers in an institution, and each of the managers may manage a plurality of users. Besides, the users may include the general elderly, dementia patients, patients with mild cognitive impairment, and the like, and may be classified into the general elderly, dementia patients, or patients with mild cognitive impairment.

In this way, as each institution registers managers and users, integrated management is possible.

The user analysis unit 230 may analyze an action of a user through location information.

In this case, the user analysis unit 230 may analyze various actions, an amount of exercise according to an action, a wandering pattern, and the like.

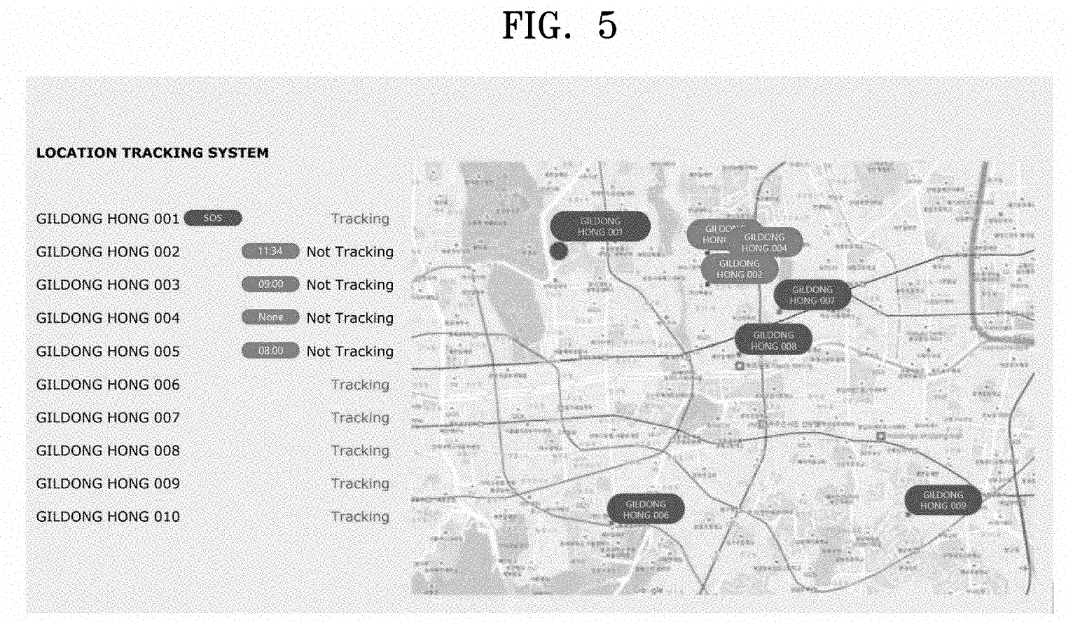

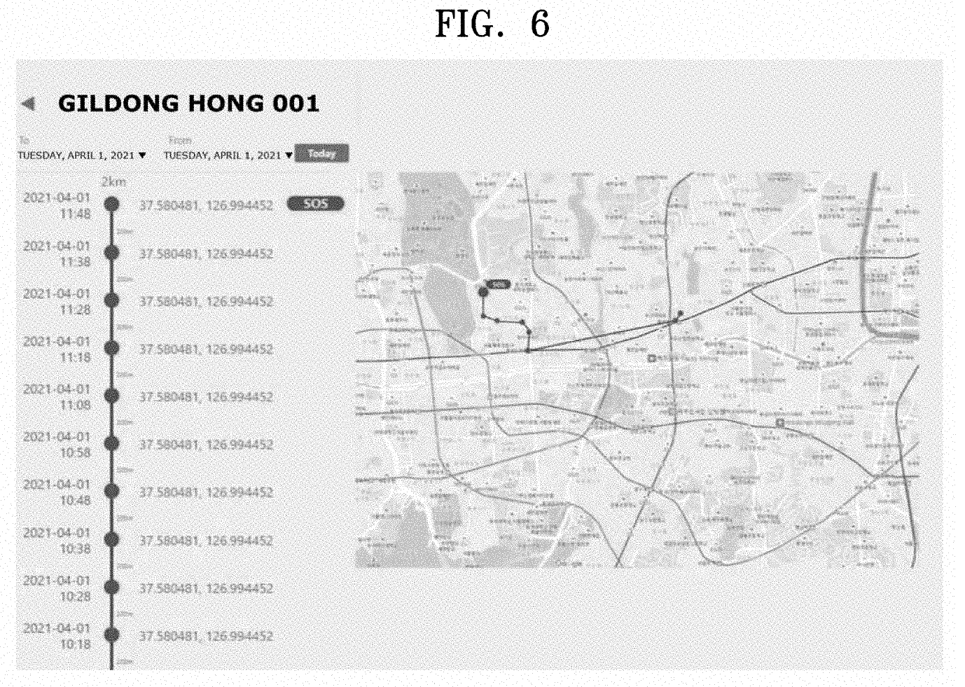

In more detail, as illustrated in FIGS. 5 and 6, the user analysis unit 230 may perform movement analysis of analyzing whether a user moves, based on the location information and moving line analysis of tracking and analyzing a moving line, along which the user wanders, through the movement analysis. In this case, the action may be analyzed by using the movement and moving line during a preset time.

Moreover, the user analysis unit 230 may further perform speed analysis of analyzing the user's walking speed through the movement analysis and the moving line analysis and transportation means analysis of analyzing whether the user is moving on foot or through a transportation means through the speed analysis.

Also, the user analysis unit 230 may extract a moving line of the user identified through the transportation means analysis, may analyze an daily exercise amount through the extracted moving line, and may analyze an average exercise amount based on at least two or more of daily exercise amounts thus already analyzed. In this case, the user analysis unit 230 may analyze whether or not the daily exercise amount is satisfied, by comparing the daily exercise amount with the average exercise amount.

Besides, the user analysis unit 230 may analyze a patient's wandering pattern through the location analysis and moving line analysis of a patient having mild cognitive impairment, and may analyze a user wandering pattern through the location analysis and moving line analysis of the user. The user wandering pattern may correspond to a normal elderly person, but is not limited thereto.

At this time, it is preferable that the patient and the user are positioned in the same safe zone, but is not limited thereto.

Furthermore, the user analysis unit 230 may analyze a stop time at a place, at which the user is stopped, through the location information.

In this way, the user analysis unit 230 may analyze various actions of the user through the location information and may not only provide the user with a warning of caution and danger through this analysis but may also call a manager, thereby preventing unforeseen accidents, as well as performing prompt check and handling.

The information management unit 240 may update real-time location information of the user and the analysis information analyzed by the user analysis unit 230 in user information registered in the information registration unit 210 and may manage the user information.

Moreover, the user information managed by the information management unit 240 may be shared such that another manager other than the manager is capable of identifying the user information and integrated management may be achieved by a plurality of managers.

Also, institutions may be operated with different purposes from one another, and a user may be registered in a plurality of institutions and may be managed for each institution. Accordingly, it is possible to reduce redundant management through integrated management of users, and to quickly take measures by sharing information with a plurality of managers when an accident occurs.

Besides, the information management unit 240 may set a safe zone, which is based on the moving line analyzed by the user analysis unit 230 or the set address of the user.

In the meantime, the dementia patient integrated management system 200 according to an embodiment of the present disclosure further includes a call unit capable of calling a manager depending on the user's action and a monitoring unit capable of identifying analysis information of the user analysis unit 230.

In the case where a user deviates from a predetermined safe distance from the safe zone, the call unit may provide a caution warning to a manager belonging to the first institution when it is determined that the user is walking, through the transportation means analysis. And, the call unit may provide the caution warning to another manager belonging to the second institution concurrently. Here, both of the first institution and the second institution may indicate the institution managing the user information of the user.

Furthermore, when a user is moving through a means of transportation, the user reaches a location within a predetermined distance, at which a return is impossible, after the caution warning, or the user fails to move to the safe zone within a predetermined first set time after the caution warning, the call unit may provide a hazard warning to the manager belonging to the first institution. And, the call unit may provide the hazard waning to another manager be belonging to the second institution concurrently. Here, both of the first institution and the second institution may indicate the institution managing the user information of the user.

That is, the call unit may provide the caution warning and/or hazard warning to all the managers managing the user, only when it is necessary to be cautious about the user such as a situation in which the user leaves the safe zone and/or cannot return to the safe zone within a certain period of time. By doing so, it is possible to efficiently process data transmission/reception for user management and to prevent a user's risk situation with a higher probability.

Here, the call unit may provide a warning type through a text message to a manager's terminal or the monitoring unit. Moreover, when receiving a caution warning call, the manager may take measures such as reviewing the user's action through the monitoring unit. When receiving a hazard warning call, the manager may take measures such as contacting the user by using the mobile terminal 100. Also, the manager receiving the caution warning or the hazard warning may take measures according to manuals, but it is not limited to the above descriptions.

Furthermore, when the daily exercise amount is less than the average exercise amount, the call unit may provide a caution warning to the manager. When the daily exercise amount is less than the minimum exercise amount among the already analyzed daily exercise amounts, the call unit may provide a hazard warning to the manager.

Moreover, the user analysis unit 230 may analyze a distance, at which the user is capable of moving per minute, through the daily exercise amount. In this way, the user analysis unit 230 may also analyze whether a means of transportation is used.

When the similarity between a patient wandering pattern and a user wandering pattern is not less than a predetermined value, the information management unit 240 may update information about mild cognitive impairment in the user information of the user.

At this time, a patient and a user are located in the same safe zone. The patient wandering pattern may be compared with the user wandering pattern. In this case, when a similar wandering pattern is formed, it may be determined that the user has mild cognitive impairment.

Here, there are delusions, hallucinations, wandering, excitement, aggression, insomnia, depression, and the like among actions of mild cognitive impairment. The user's mild cognitive impairment may be determined by comparing the patient with the user through a wandering pattern among them.

In addition, the wandering pattern may be obtained by comprehensively patterning a wandering action or may indicate that a user repeatedly goes and stops. At this time, when the patient and the user are located in the same safe zone, a wandering pattern according to mild cognitive impairment may appear similarly depending on the same environment. Accordingly, when the patient wandering pattern is compared with the user wandering pattern in the same environment, the mild cognitive impairment may be determined accurately.

At this time, the call unit may select a call case through user information about a user. When it is determined that the user has mild cognitive impairment, the call unit may provide the manager with a hazard warning instead of a caution warning.

In the meantime, the dementia patient integrated management system 200 according to an embodiment of the present disclosure may further include an environment information receiving unit, a check unit, a path extraction unit, and a search information transmitting unit.

When a stop time reaches a predetermined second set time, the environment information receiving unit may request and receive environment information included in the location information of a place, at which a user is stopped, from the Korea Meteorological Administration.

At this time, the environment information provided from the Korea Meteorological Administration may be temperature and air quality information, but is not limited thereto.

Besides, when the stop time is not less than a predetermined third set time, the call unit may provide the manager with a caution warning. When the temperature is out of a predetermined temperature range or the air quality value is out of a predetermined air quality value in the environment information received by the environment information receiving unit, the call unit may provide the manager with a caution warning.

In particular, when the user's underlying disease is updated in the user information, the call unit may provide the manager with a caution warning by calculating the air quality value at a rate lower than that in the case of a user without underlying diseases by about 30 to 50%.

When a caution warning and/or hazard warning is received from the call unit, the check unit attempts to make a phone call between the manager's terminal and the user's mobile terminal 100. At this time, in addition to the manager's terminal, the check unit may attempt to make a phone call with an automatic answering function.

For example, the check unit attempts to make a phone call to the user's mobile terminal 100 through the automatic answering function. At this time, predetermined comments are output by the display unit or sound output module of the mobile terminal 100 of a user, the user may respond through an input unit depending on the comments.

Here, when an error occurs in the user's response during a predetermined number of times, the call unit may request the manager to go into action.

When the phone call attempted through the check unit is not connected, the path extraction unit determines that a user is missing, and extracts a missing search path. The path extraction unit extracts a past user wandering pattern identified within a predetermined search distance, based on the latest location information of the missing person who is determined to be missing. In other words, the path extraction unit extracts a missing search path within a predetermined search distance based on a past user wandering pattern of the user. In this case, when a missing person is not capable of being found through the search, the search distance may be extended.

The search information transmitting unit delivers place information of a stop place in the user wandering pattern extracted by the path extraction unit to the searcher, such that a searcher intensively searches for the missing person at a location corresponding to the place information. That is, it is estimated that the stop place is a place frequently visited by the user. As the stop place is found intensively, it is possible to quickly find witnesses, thereby helping the search.

Furthermore, the search information transmitting unit may transmit a photo and characteristic information of the missing person to at least one or more searcher terminals.

At this time, the searcher terminal is a delivery person's terminal that has an agreement with an institution and is positioned in a specific radius based on the stop place. In addition, the delivery person is a person that has an agreement with an institution, and may be a rider for a delivery app or a restaurant.

Moreover, when the photo or video of a missing person is captured, the searcher terminal may send the photo or video to the manager's terminal. When it is identified that the missing person is correct, by the manager's terminal, the searcher terminal may automatically delete the photo and characteristic information of the missing person. This is used to protect personal information. It is desirable to completely delete the photo and the characteristic information of the missing person from the searcher terminal sending the photo and the characteristic information of the missing person.

As such, it is possible to quickly search for a specific region, by requesting a search through a delivery person who specializes in delivery in the specific area. Besides, when a missing person is found while a manager goes into actions, the manager may quickly move to the found place and may take over the missing person.

When the missing person is taken over to the manager, a reward delivery unit may deliver the agreed reward to the searcher terminal that finds the missing person.

Meanwhile, another embodiment of a dementia patient integrated management and dementia determination system 200 according to an embodiment of the present disclosure will be described.

Referring to FIGS. 3 and 4, the dementia patient integrated management and dementia determination system 200 according to an embodiment of the present disclosure includes the information registration unit 210, the question providing unit 250, the information receiving unit 220, and the user analysis unit 230.

Manager information of a manager belonging to at least one or more institutions and user information of a user managed by the manager are registered in the information registration unit 210.

The question providing unit 250 generates a cognitive training question and then provides the generated cognitive training question to the user.

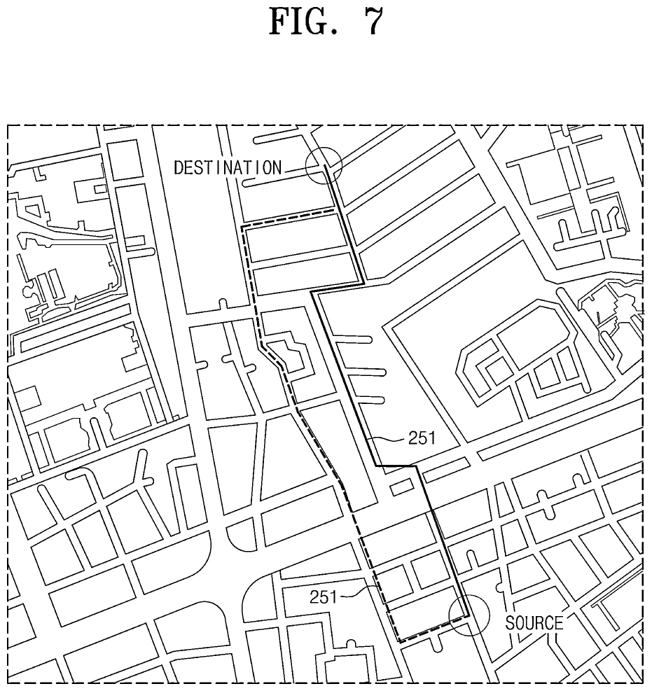

Here, when generating a cognitive training question, the question providing unit 250 may extract a plurality of movement paths, through which an actual user moves, through real-time location information of the user and may generate one or more of the extracted movement paths as a cognitive training path 251 as shown in FIG. 7.

Furthermore, it is preferable that the cognitive training question corresponds to the cognitive training path 251, through which the user is capable of moving from a source to a destination, within a moving radius in which the user is capable of moving on foot, but is not limited thereto.

Moreover, the cognitive training question may include a question of providing only a source and destination depending on a user's dementia level or a question of providing both source and destination paths.

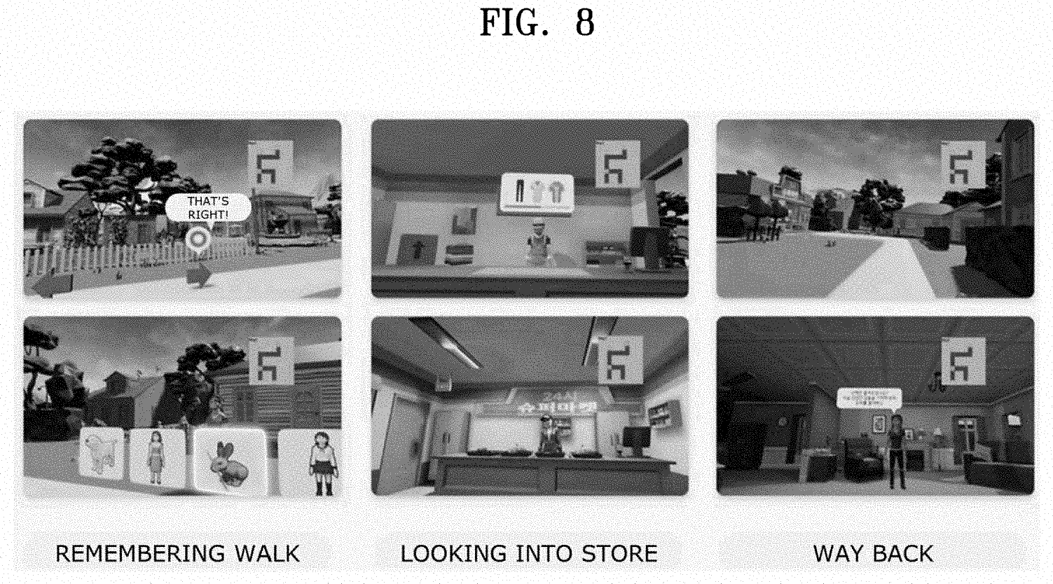

Also, referring to FIG. 8, the cognitive training question may include a variety of missions, such as remembering a walk, looking into a store, or the like while a user moves along the cognitive training path 251. Accordingly, the missions may be accomplished. The user's dementia level may be analyzed depending on whether these missions have been accomplished correctly.

In the meantime, the movement path may be a path, on which the user moves once, or a path on which the user moves multiple times. Besides, a waypoint may be included in the middle of a movement path.

Accordingly, when extracting the movement path, first of all, the question providing unit 250 sets a source and a destination and extracts an expected path on which a user is capable of moving from the set source to the set destination. Then, the question providing unit 250 extracts the same extraction path by comparing the expected path with the movement path. In addition, the question providing unit 250 may finally extract the movement path, which satisfies one or more of a first condition of moving a predetermined number of times and a second condition that the number of paths for moving from a source to a destination is not greater than the predetermined number of paths, from among the extraction paths.

Here, it may be described that the predetermined number of times is three or more, and the predetermined number of paths is three or less, but is not limited thereto. Because there is a high probability that a user is sufficiently aware of surroundings on a movement path on which the user repeatedly takes a walk three times or more, the user may perform cognitive training questions easily and familiarly. Moreover, because there are many paths of which the number exceeds three, it is difficult to identify and determine whether the user performs the cognitive training question.

As such, when the movement path is extracted, the movement path may consist of one or more paths.

Accordingly, the cognitive training question includes one or more of the cognitive training paths 251, of which the number is equal to or less than a predetermined number of paths. In particular, it is desirable that the cognitive training question includes two or more of the cognitive training paths 251. For example, the reason is that the degree of dementia is capable of being evaluated negatively, when a user deviates from the predetermined cognitive training path 251, in the case where the question is conducted by using one the cognitive training path 251.

The information receiving unit 220 receives the user's real-time location information.

The user analysis unit 230 analyzes a performance result according to execution of the user's cognitive training question through the location information and evaluates a dementia degree of the user depending on the performance result.

Furthermore, while the user is conducting a cognitive training question, the user analysis unit 230 may analyze whether an actual path on which the user has moved matches and the cognitive training path 251.

Here, the user's dementia degree may be evaluated depending on the match.

Moreover, when the user's action includes an unusual action while the user is conducting a cognitive training question, the user analysis unit 230 may subtract a score corresponding to the unusual action from a predetermined score. When the user moves to a destination of the cognitive training path 251, the user analysis unit 230 may analyze the degree of dementia through the subtracted score.

Here, the unusual action may include an action, in which a user repeatedly moves on a partial path, and an action in which the user's location deviates from the cognitive training path 251 by a specific radius or more.

In the meantime, the dementia patient integrated management and dementia determination system 200 according to an embodiment of the present disclosure may further include a call unit, a check unit, and a search information transmitting unit.

When the predetermined score is subtracted and then the subtracted score is within a specific range while the user conducts a cognitive training question, the call unit calls a manager.

At this time, when the predetermined score is subtracted and the subtracted score is within a predetermined first score range, the call unit may provide a caution warning to the manager. When the subtracted score is decreased to be lower than a score within the first score range, and the subtracted score is within the predetermined second score range, the call unit may provide a hazard warning to the manager.

When the caution warning or hazard warning is received from the call unit, the check unit may attempt to make a phone call between the manager's terminal and the user's mobile terminal 100. At this time, the check unit determines that the user is a missing person when the phone call is not made.

The search information transmitting unit may deliver, to a helper, place information including real-time location information of the user received while a cognitive training question is conducted, such that the helper intensively searches for the missing person at a location corresponding to the place information.

Furthermore, the search information transmitting unit may transmit a photo and characteristic information of the missing person to at least one or more helper terminals.

In this case, the helper terminal is a terminal of a delivery person, which has an agreement with an institution and is positioned in a specific radius based on location information. When a photo or video of the missing person is captured, the helper terminal transmits the photo or video to the terminal of the manager and automatically deletes the photo and characteristic information of the missing person when it is identified that the missing person is correct, by the terminal of the manager.

When the missing person is taken over to the manager, a reward thus previously agreed may be delivered to the helper terminal that finds the missing person.

Those skilled in the art to which the present disclosure pertains will understand that the present disclosure may be embodied in other specific forms without changing the technical spirit or essential characteristics thereof. Therefore, the above-described embodiments are examples in all aspects, and should be construed not to be restrictive. The scope of the present disclosure is defined by claims to be described below rather than the detailed description, and it should be interpreted that the scopes or claims of the present disclosure and all modifications or changed forms derived from the equivalent concept are included in the scopes of the present disclosure. Besides, the operation sequence of the configuration described in the above process does not necessarily need to be performed in time-series order. When the gist of the present disclosure is satisfied even though each configuration and the execution order of steps are changed, it is obvious that such the process belongs to the scope of the present disclosure.

As described above, an effect of the present disclosure may provide a dementia patient integrated management system capable of collectively determining and training cognitive abilities through pieces of user information by registering and managing manager information of a manager belonging to at least one or more institutions and user information of a user managed by the manager, and by sharing the user information such that another manager other than the manager is capable of identifying the user information.

Moreover, an effect of the present disclosure may provide a dementia patient integrated management system capable of promptly responding to abnormal symptoms such as disappearance of dementia patients and departure from a safe zone, by variously analyzing a user's action by using location information and identifying environment information.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A dementia patient integrated management system, the system comprising:
an information registration unit configured to register manager information of a manager belonging to at least one institution and user information of a user managed by the manager;
an information receiving unit configured to receive real-time location information of the user;
a user analysis unit configured to analyze an action of the user through the location information; and
an information management unit configured to update the real-time location information of the user and analysis information analyzed by the user analysis unit in the user information registered in the information registration unit and to manage the user information,
wherein the user information managed by the information management unit is shared such that another manager other than the manager is capable of identifying the user information, and
wherein integrated management is achieved by a plurality of managers.

2. The system of claim 1, wherein the user analysis unit analyzes an action during a predetermined time through movement analysis of analyzing whether the user moves, based on the location information and moving line analysis of tracking and analyzing a moving line, along which the user wanders, through the movement analysis.

3. The system of claim 2, wherein the information management unit sets a safe zone, which is based on the moving line analyzed by the user analysis unit or a set address of the user, and
wherein another manager manages the user information of the user,
wherein the user analysis unit further performs speed analysis of analyzing a walking speed of the user through the movement analysis and the moving line analysis and transportation means analysis of analyzing whether the user is moving on foot or through a transportation means, through the speed analysis,
further comprising:
a call unit configured to:
when the user deviates from a predetermined safe distance from the safe zone,
provide a caution warning to the manager and another manager when it is determined that the user is walking, through the transportation means analysis; or
provide a hazard warning to the manager and another manager when the user is moving through the transportation means, the user reaches a location within a predetermined setting distance, at which a return is impossible, after the caution warning, or the user fails to move to the safe zone within a predetermined first set time after the caution warning.

4. The system of claim 3, wherein the user analysis unit extracts the moving line, on which the user is walking and which is identified through the transportation means analysis, analyzes an daily exercise amount through the extracted moving line, analyzes an average exercise amount based on at least two daily exercise amounts thus already analyzed, and analyzes whether the daily exercise amount is satisfied, by comparing the daily exercise amount with the average exercise amount, and
wherein the call unit provides the caution warning to the manager when the daily exercise amount is not satisfied, and provides the hazard warning to the manager when the daily exercise amount is less than a minimum exercise amount among the daily exercise amounts thus already analyzed.

5. The system of claim 3, wherein the user analysis unit analyzes a patient wandering pattern through location analysis and moving line analysis of a patient having mild cognitive impairment, and analyzes a user wandering pattern through location analysis and moving line analysis of the user,
wherein, when a similarity between the patient wandering pattern and the user wandering pattern is not less than a predetermined value, the information management unit updates mild cognitive impairment information in the user information of the user, and
wherein the call unit selects a call case through the user information about the user, and provides the manager with the hazard warning instead of the caution warning when it is determined that the user has mild cognitive impairment.

6. The system of claim 5, wherein the user analysis unit analyzes a stop time at a place, at which the user is stopped, through the location information,
further comprising:
an environment information receiving unit configured to request and receive environment information included in location information of the place, at which the user is stopped, from Korea Meteorological Administration when the stop time reaches a predetermined second set time,
wherein the call unit provides the manager with the caution warning when the stop time is not less than a predetermined third set time, and provides the manager with the caution warning when a temperature in the environment information received by the environment information receiving unit is out of a predetermined temperature range or an air quality value in the environment information is out of a predetermined air quality value.

7. The system of claim 6, further comprising:
a check unit configured to attempt to make a phone call between a terminal of the manager and a mobile terminal of the user when the caution warning or the hazard warning is received from the call unit;
a path extraction unit configured to determine disappearance, when the phone call attempted through the check unit is not connected, to extract a missing search path, and to extract a past user wandering pattern identified within a predetermined search distance, based on latest location information of a missing person who is determined to be missing; and
a search information transmitting unit configured to deliver place information of a stop place in the user wandering pattern extracted by the path extraction unit to a searcher such that the searcher intensively searches for the missing person at a location corresponding to the place information.

8. The system of claim 7, wherein the search information transmitting unit transmits a photo and characteristic information of the missing person to at least one searcher terminal,
wherein the searcher terminal is a terminal of a delivery person, which has an agreement with the institution and which is positioned in a specific radius based on the stop place, and
wherein, when a photo or video of the missing person is captured, the searcher terminal transmits the photo or video to the terminal of the manager and automatically deletes the photo and characteristic information of the missing person when it is identified that the missing person is correct, by the terminal of the manager,
further comprising:
a reward delivery unit configured to deliver an agreed reward to the searcher terminal, which finds the missing person, when the missing person is taken over to the manager.
